(19) United States
(12) Patent Application Publication
(10) Pub. No.: US 2025/0288344 A1
(43) Pub. Date: Sep. 18, 2025

(54) ELECTRICAL SURGICAL TOOLS FOR CIRCUMCISION

(71) Applicant: SafeCirc, LLC, Wakefield, RI (US)

(72) Inventor: David Tomlinson, Wakefield, RI (US)

(21) Appl. No.: 18/860,527

(22) PCT Filed: Apr. 26, 2023
(86) PCT No.: PCT/US2023/066251

§ 371 Date: Oct. 25, 2024

Related U.S. Application Data
(60) Provisional application No. 63/335,034, filed on Apr. 26, 2022.

(51) Int. Cl. A61B 18/08; A61B 18/00

(52) U.S. Cl. CPC A61B 18/082; A61B 2018/00077; A61B 2018/00601

(57) ABSTRACT

A male circumcision device includes a ring component and a cutting component. The ring component includes a base portion and a shaft. and an electrode disposed on the base portion or the shaft. The cutting component includes a body defining an inner aperture and a cutting implement having an electrically-conductive portion disposed at a distal end of the cutting component. The device also includes a power source for applying an electrical current through the electrode and through the electrically-conductive portion of the cutting component when the cutting component is coupled to the ring component during a cutting operation.

### CROSS-REFERENCE TO RELATED APPLICATION

This application claims the benefit of priority to U.S. provisional application 63/335,034, filed on Apr. 26, 2022, which application is hereby incorporated by reference in its entirety.

### FIELD OF THE DISCLOSURE

The present disclosure generally relates to medical devices that use energy in the form of electricity to make an incision, seal tissue, and obtain hemostasis while performing a male circumcision operation.

### BRIEF DESCRIPTION OF THE DRAWINGS

FIG. 1 is a perspective diagrammatic view of an example device for performing an electrosurgical incision in a male circumcision operation.

FIG. 2 is a cross-sectional view of a portion of the device of FIG. 1, taken along line 2-2 in FIG. 1.

FIG. 3 is a cross-sectional view of a portion of the device of FIG. 1, taken along line 3-3 in FIG. 1.

FIGS. 4A-4C illustrate various stages of a male circumcision operation using the device of FIG. 1.

FIGS. 5A and 5B illustrate an example device for performing an incision in a male circumcision operation with an inductively-heated blade.

FIG. 6 is an exploded view of an example device for performing an electrosurgical incision in a male circumcision operation, which device may be readily combined with the electrical components of FIGS. 1-5.

FIG. 7 is a cross-sectional view of of the device of FIG. 6 in the opened or relaxed position.

FIG. 8 is a cross-sectional view of of the device of FIG. 6 in the closed or engaged position.

FIG. 9 illustrates an example ring component of the device of FIG. 6.

FIGS. 11, 12, 13, and 14 illustrate the ring component of FIG. 9 being inserted into the foreskin of the neonatal penis.

FIG. 15 illustrates a method of performing a male circumcision with a device according to the present disclosure.

### DETAILED DESCRIPTION

U.S. Pat. No. 8,353,918 (“'918 Patent”), which is hereby incorporated by reference in its entirety, discloses a method of using a disposable neonatal circumcision device that secures the foreskin of the penis in a precise location, applies radially circumferential clamping, and delivers a longitudinal circumferential cutting device along the path precisely controlled by the device itself, not the operator, ensuring the incision to the clamped foreskin is made in the precise location, independent of the operator.

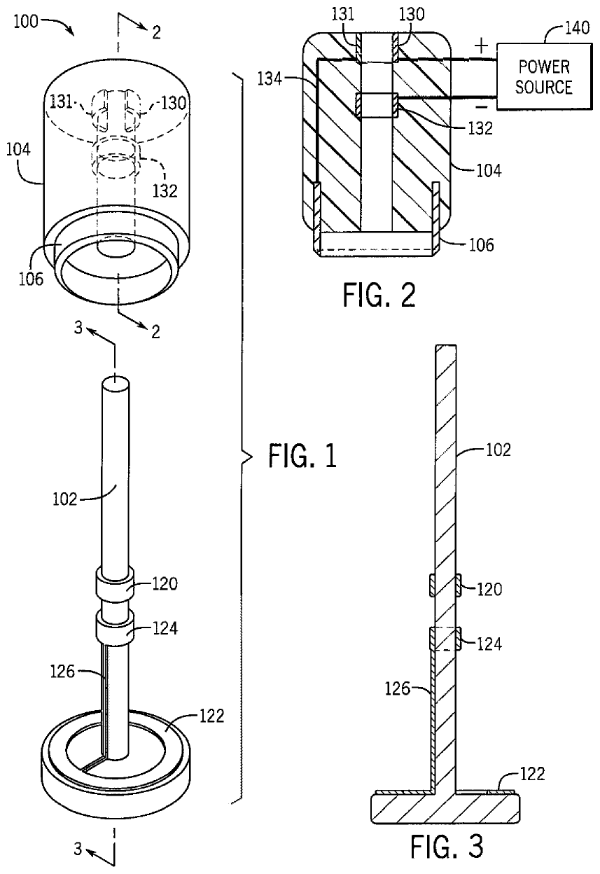

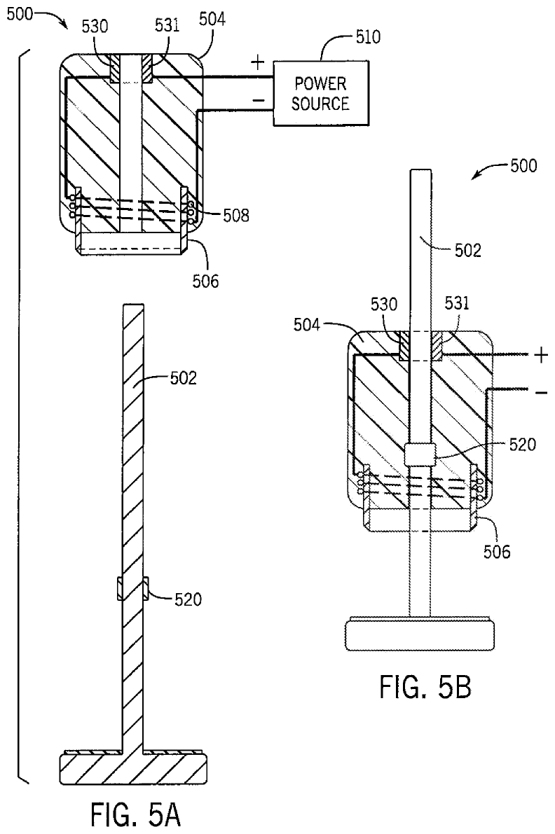

The instant disclosure includes devices and methods for performing electrically-enhanced male circumcision, for example as improvements to the devices of the '918 Patent. In a first example discussed below with respect to FIGS. 1-4C, an electrical current may be applied between a ring component and a clamping-cutting device. The current may flow through the foreskin during the incision, resulting in a cleaner and more consistent incision, sealing the tissue, and resulting in better hemostasis. In a second example, discussed below with respect to FIGS. 5A and 5B, a blade of the clamping-cutting device may be inductively heated and may therefore cauterize the incision site as the incision is made, sealing the tissue and obtaining better hemostasis. In both examples, an electrical current is applied through various components for enhancing the safety and efficacy of the circumcision operation.

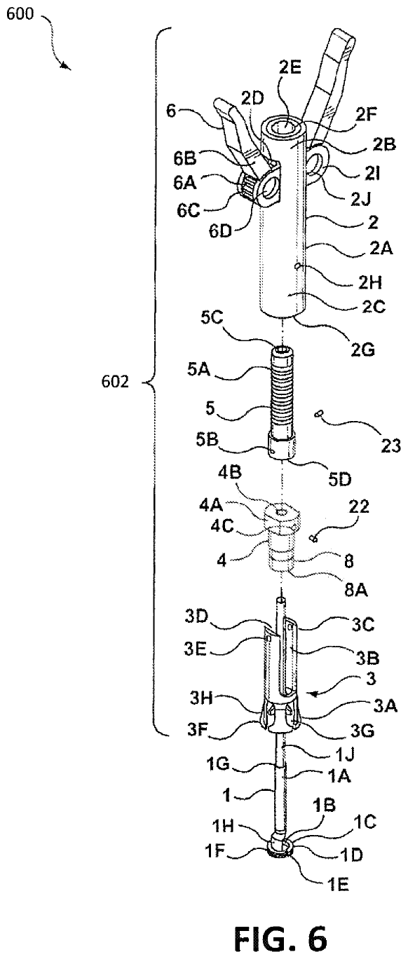

Referring to the drawings, wherein like reference numerals refer to the same or similar features in the various views, FIG. 1 is an exploded diagrammatic perspective view of an example tool 100 for performing an electrosurgical incision during male circumcision. The tool 100 may include a ring component 102 and a cutting component 104 with a blade 106. FIG. 2 is a cross-sectional view of the tool cutting component 104, taken along line 2-2 in FIG. 1, and FIG. 3 in a cross-sectional view of the ring component 102, taken along line 3-3 in FIG. 1. The ring component 102 may be structurally similar to the “ring component 1” or the “ring component 112” of the '918 Patent or of “ring component 1” of FIGS. 6-15 herein, for example, including the associated broader structure of which the ring component 102 may be a part. The cutting component 104 may be structurally similar to the “blade holder 4” or “clamping-cutting device 200” of the '918 Patent or: blade holder 4″ of FIGS. 6-15 herein, for example, including the associated broader structure of which the cutting component 104 may be a part. The blade 106 may be structurally similar to the “blade 8” or “blade 204” of the '918 Patent or the “blade 8” of FIGS. 6-15 herein, for example, including the associated broader structure of which the blade 106 may be a part. Alternatively, the blade 106 may be a cutting element that cuts solely though transmission of electricity, rather than through forceful application of a sharpened edge. Relative to the structures disclosed in the '918 Patent, the tool 100 may include electrical components for applying an electrosurgical incision during a male circumcision operation, as discussed below.

Referring to FIGS. 1-3, the ring component 102 may include three electrodes 120, 122, 124, and an electrical coupling 126 from the electrode 122 to the electrode 124. The cutting component 104 may include three electrodes 130, 131, 132, and an electrical coupling 134 from the electrode 131 to the blade 106. As will be described below, the blade 106 may also function as an electrode during an incision.

The electrodes 120, 124 may be or may include circumferential bands around an exterior surface of a primary cylinder of the ring component 102. The electrodes 120, 124 may have surface areas that are parallel with the axis of the ring component 102 and radially- outward facing. The electrode 122 may be or may include a circular band disposed on a cutting surface of the ring component 102. The electrode 122 may have a surface area that is perpendicular to the axis of the ring component.

The electrodes 130, 131 may be or may include opposed arcuate plates. The electrode 132 may be or may include a circumferential band. The electrodes 130, 131, 132 may be disposed on an inner surface of the cutting component 104, e.g., on a surface of an axial through-bore of the cutting component. The electrodes 130, 131, 132 may have respective surface areas that are parallel with the axis of the ring component 102 and facing radially-inward. The electrodes 120, 124 may be placed respective axial distances above the electrode 122 that is equal, or nearly equal, to the respective axial distances of the electrodes 130, 132 above the tip of the blade 106. Electrode 120 may serve as a safety switch. Positive current can only pass from electrode 130 to electrode 131 when the ring component 102 is inserted into the bore of the cutting component 104, bringing the electrode 120 in line with electrodes 130, 131 to complete the circuit. Current can only pass to the blade 106 of the cutting component 104 when the ring component 102 is appropriately positioned in relation to the cutting component 104, ensuring that current can only flow when the two parts are being used appropriately. This safety mechanism is intended to protect the clinician and the patient during use of the electrosurgical current.

The electrodes 120, 122, 124, 130, 132 and electrical couplings 126, 134 may include or may be made of electrically-conductive material, such as metal.

An electrical power source 140 may be electrically coupled across the electrodes 130, 132 of the cutting component 104. For example, a positive terminal of the power source 140 may be coupled to the electrode 130, and a negative terminal of the power source 140 may be coupled to the electrode 132. The power source 140 may output an electrical signal of a desired voltage, current, and/or frequency for creating an effective circumcision.

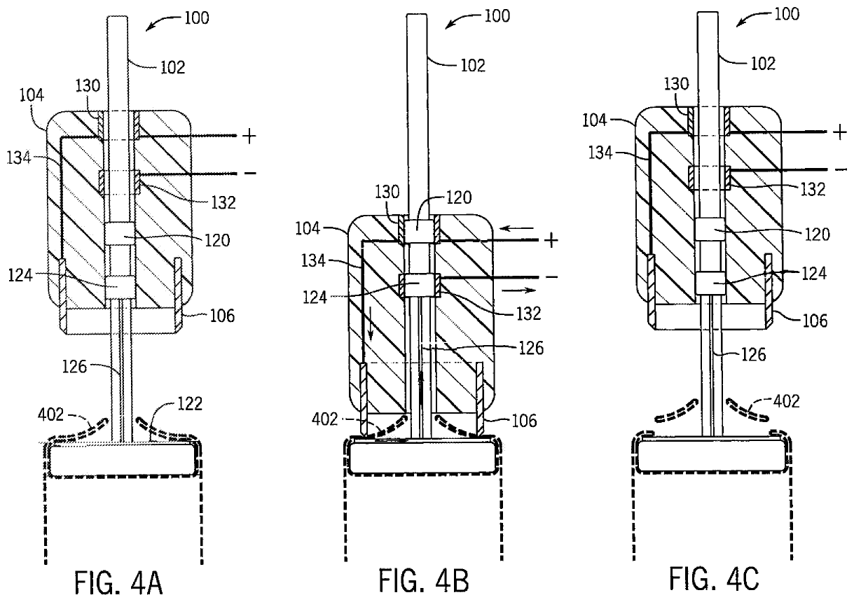

FIGS. 4A-4C illustrate various states of use of the device 100. FIG. 4A illustrates a pre-incision state, FIG. 4B illustrates an incision state, and FIG. 4C illustrates a post-incision state. During a male circumcision operation, the cutting component 104 may be placed about the ring component 102 and advanced axially down the ring component 102 from the position of FIG. 4A towards the position of FIG. 4B with the foreskin 402 placed on and about the cutting surface electrode 122. As the cutting component reaches an axial position where the blade 106 contacts the foreskin 402, the electrode 130 reaches the same axial position as the electrode 120, and the electrode 132 reaches the same axial position as the electrode 124, as shown in FIG. 4B. A complete circuit may thus be formed that includes the electrode 130, electrical coupling 134, blade 106, electrode 122, electrical coupling 126, electrode 124, and electrode 132. The power source may be activated, and current may flow through the complete circuit. As shown in FIG. 4B, the foreskin 402 may be disposed between the electrode 122 and the blade 106, and current may therefore flow through the foreskin 402 as pressure is applied to the blade 106 to cut the foreskin. When the cutting component 104 is moved axially away from the electrode 122, the circuit is broken and electrical current flow stops, even if the power source remains on.

In some embodiments, the power source may be activated before the blade contacts the skin to ensure cautery action to achieve hemostasis. In some embodiments, the clinician using the device 100 may connect the device 100 to the power source 140 and activate the power source 140. As the cutting component 104 moves down the ring component 102 to a position in which the blade contacts the foreskin, current flow begins. In another embodiment, a ring electrode may be used instead of a sharpened blade, the clinician may advance the ring electrode to the foreskin. Once in place, the clinician may activate the power source (e.g., with a foot switch).

FIG. 5A is an exploded cross-sectional view of an example surgical device 500 for performing an incision in a male circumcision operation with an inductively-heated blade, and FIG. 5B is a partial cross-sectional view of the tool 500.

The tool 500 may include a ring component 502 and a cutting component 504 with a blade 506. The ring component 502 may be structurally similar to the “ring component 1” or the “ring component 112” of the '918 Patent, for example, including the associated broader structure of which the ring component 502 may be a part. The cutting component 504 may be structurally similar to the “blade holder 4” or “clamping-cutting device 200” of the '918 Patent, for example, including the associated broader structure of which the cutting component 504 may be a part. The blade 506 may be structurally similar to the “blade 8” or “blade 204” of the '918 Patent, for example, including the associated broader structure of which the blade 506 may be a part. Relative to the structures disclosed in the '918 Patent, the tool 500 may include electrical components for inductively heating the blade 506 for an incision during a circumcision operation, as discussed below.

The cutting component 504 may include an embedded coil 508 that may be selectively coupled to a power source 510. When an electrical signal is applied to the coil 508, the coil 508 may generate a magnetic field. In turn, the blade 506 may be heated by the magnetic field generated by the coil 508. Accordingly, the blade 506 may include or may be made from an appropriate inductively-heatable material, such as a ferrous material. During a circumcision operation, the blade 506 may be inductively heated to an appropriate temperature (e.g., 800 degrees Fahrenheit or more) to cauterize the incision site as the incision is being made. In embodiments, the cutting component 504 and/or power source 510 may include one or more features for ensuring that the blade 506 is heated to a desired temperature before the blade 506 contacts the foreskin, and/or for ensuring that the blade 506 does not rise to an unacceptable temperature. For example, a thermocouple or other temperature sensor may be coupled to the blade 506, and the output of the thermocouple or other temperature sensor may be output to a controller and/or the power source 510 to ensure that the power source 510 is activated to bring the temperature of the blade 506 to an appropriate temperature and remain at that appropriate temperature.

The cutting component 504 may further include two electrodes 530, 531, and the ring component 502 may include an electrode 520. The electrodes 520, 530, 531 may be similar to and serve a collectively similar function to the electrodes 120, 130, 131. That is, electrode 520 may serve as a safety switch. Positive current can only pass from electrode 530 to electrode 531 when the ring component 502 is inserted into the bore of the cutting component 504, bringing the electrode 520 in line with electrodes 530, 531 to complete the circuit. Current can only pass to the coil 508 of the cutting component 504 when the ring component 502 is appropriately positioned in relation to the cutting component 504, ensuring that current can only flow when the two parts are being used appropriately.

The electrical components and methods of FIGS. 1-5B may be used in combination with the structures and methods of FIGS. 6-15. That is, as described below, electrodes and other electrically conductive elements, and their use in a circumcision, as illustrated in and described with respect to FIGS. 1-5B may be incorporated into the structure and methods of FIGS. 6-15.

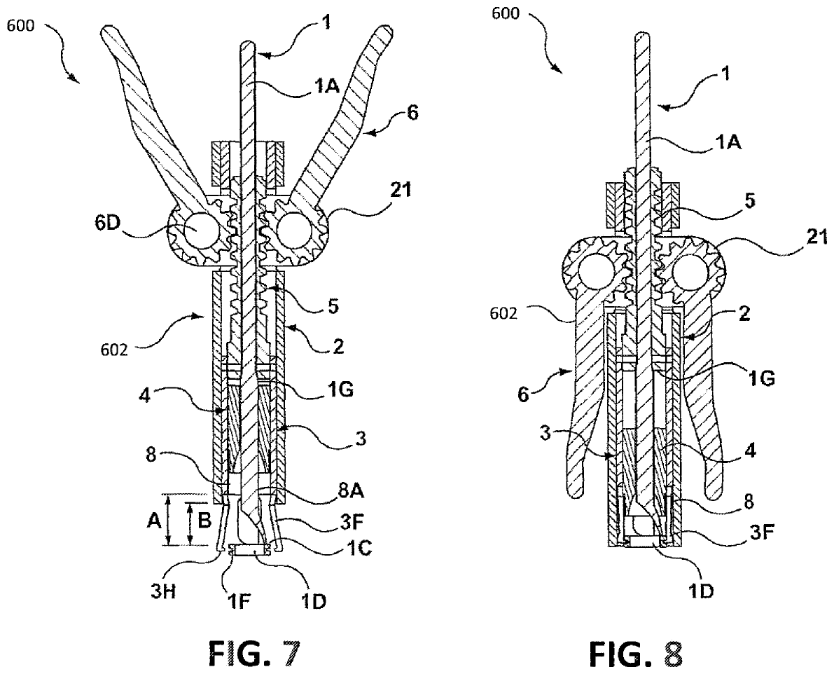

FIGS. 6, 7, and 8 illustrate an example embodiment of a male circumcision device 600, the structure of which may serve as a base structure for the device 100. The device 600 includes ring component 1 and clamping-cutting device 602. Clamping-cutting device 602 includes operably connected housing 2, clamping member 3 with retractable arms 3F, blade holder 4 with integral blade 8, gear track 5, and two lever arms 6. Clamping member 3 is operably connected to lever arms 6 such that when lever arms 6 are actuated up retractable arms 3F are positioned out of clamping-cutting device 102 to the opened or resting position (FIG. 7) or when lever arms 6 are actuated down the clamping-cutting device 102 traverses over the clamping member 3 to the closed or engaged position (FIG. 8).

In the open position (FIG. 7), the ring component 1 is free to move into and out of clamping-cutting device 602. As lever arms 6 move downward to the closed position (FIG. 3), retractable arms 3F of clamping member 3 radially close and apply substantially even circumferential pressure to open ring ID of the ring component 1 to firmly close the ring ID and hold the ring component 1 in place as the housing 2 of the clamping-cutting surface 602 is advanced over the retractable arms 3F. In the clamped configuration, the ring component 1 and the clamping-cutting device 602 act as a single, integral component controlled by the actuation of level arms 6. Downward progress of crushing- cutting component 602, a distance designated B, advances housing 2 over retractable arms 3F such that the clamping operation is fully activated or engaged. At the moment the housing 2 has been advanced the distance B and the crush has been completed, the blade has traveled the same distance B and is positioned above the ring and cutting surface. It is not until the lever arm 6 has been further actuated and the housing 2 and blade 8 have been advanced further down clamping member 3 a distance designated A that the cutting edge 8A is brought into contact with the cutting surface 1C of ring ID thereby incising the foreskin positioned between the cutting edge 8A and the cutting surface 1C. During further advancement of housing 2 over the retractable arms the crushing force of retractable arms 3F are maintained by the inner diameter of the housing 2. The timing of the crush and cut is controlled by distances A and B. These distances are sufficiently different to ensure that the clamping action occurs prior to the incision. And more importantly, that an incision can not possibly be made without first activating and maintaining the clamping action. Downward progress A of crushing-cutting component 602 stops when the cutting edge 8A of blade 8 contacts the top or cutting surface 1C of ring ID of the ring component 1. The sequence of the crush and cut is controlled by the device itself when the operator actuates the lever arms downward fully in one single motion. As lever arms 6 are actuated, the present invention clamps the foreskin radially to create a substantially symmetrical and even circumferential hemostasis at the axial position and at the same time as the motion continues and only after the crush has occurred, delivers the cutting edge of blade 8 to the cutting surface 1C of the ring ID completing the incision slightly above where the clamping member 3 crushed the foreskin and achieved the hemostasis effect. The incision to the foreskin is made while the foreskin is within housing 2 and can be out of the direct line of sight of the operator if housing 2 is made of a non-transparent material or colored.

Housing 2 is generally cylindrical, hollow, and vertically longitudinal having a top portion 2B and a bottom portion 2C. Housing 2 includes top portion 2B, bottom portion 2C, opposing slots 2D, through bore 2E, top opening 2F, bottom opening 2G, assembly hole 2H, and two pairs of projections 21. Bore 2E includes a sufficiently sized inner surface and length to receive therein clamping member 3, blade holder 4, gear track 5, and ring component 1.

Two lever arms 6 are pivotally joined to top portion 2B about a pivotal axis. Each lever arm 6 has a predetermined length and width sufficient to sustain the forces of clamping and cutting. Semi-circular gears 6A are adapted to the distal end 6B of each lever arm 6 and a handle at the other end. Lever arms 6 are positioned on opposite sides of top portion 2B of housing 2 and positioned such that a portion of semi-circular gear 6A extends through a slot 2D in housing 2 so as to engage gear track 5 within housing 2. Each semi-circular gear 6A includes a plurality of gear teeth 6C. Width of lever arms 6 are sized to fit between pair of projections 21 and are pivotally attached to projections 21 by a conventional joining device (not shown) such as bolt/nut or bushing or pressed fit pin connection. Each projection 21 may include hole 2J to receive the conventional joining device. Each semi-circular gear 6 A may include a through bore 6D to receive the conventional joining means there through pivotally connecting each lever arm 6 and projection 21 with one conventional joining means. Alternatively, two conventional joining means can be inserted through each projection 21 into semi-circular gears 6A, either with or without a through bore, to form the pivotal connection. Projections 21 act as a pivotal axis for lever arms 6 and allow lever arms 6 to rotate or pivot about an axis. Pivotal movement of lever arms 6 causes rotation of semi-circular gears 6 A to impart reciprocating movement to gear track 5. Semi-circular gears 6 A have a diameter that is sufficient to linearly move housing 2 up and down over retractable arms 3F.

Bottom opening 2G of bottom portion 2C of housing 2 has an inside diameter that is made to accommodate the top portion of clamping member 3. As housing 2 is advanced down over clamping member 3, the inside diameter of housing 2 causes retractable arms 3F of clamping member 3 to radially close, such that when housing 2 is advance completely over clamping member 3, retractable arms 3F are closed and in a position to engage open ring ID and exert a significant compressive force on grooved outer surface IF of open ring ID.

Gear track 5 includes a plurality of gear teeth 5A evenly spaced along the length of gear track 5. Gear track 5 further includes through radial hole 5B for connecting clamping member 3 to gear track 5 (disclosed in detail below). Gear track 5 is initially positioned within top portion 2B of housing 2 and is capable of axial movement within housing 2 in either longitudinal direction. Gear teeth 5 A of gear track 5 cooperate with gear teeth 6C of semi-circular gears 6A to translate gear track 5 axially within bore 2E of housing 2. Gear track 5 has a vertical passageway 5C extending through its axial center that is sized to receive only the narrow portion of shaft IA of ring component 1. By accommodating only the narrow part of shaft IA, bottom face 5D contacts or rests on notch, ledge or shoulder IG of shaft IA, and therefore aligns the clamping-cutting device 602 with ring component 1 and open ring ID (discussed in detail below). Shoulder IG can be formed by shaft IA having two sections with different diameters: an upper section IJ having a diameter smaller than diameter of 5C and a lower section IA having a diameter larger than diameter of 5C. Gear track 5 is freely rotatable about its axis and gear teeth 5A extend circumferentially around gear track 5. The contact at any given time between gear teeth 6C of semi-circular gear 6A and gear teeth 5A on gear track 5 is limited to a single tooth and preferably a single point contact.

Blade holder 4 includes circular blade 8 and blade support 4A. Circular blade 8 and blade support 4A are connected by conventional means including, for example, snap fit, press or interference fit, cooperating male/female threaded members, screw, bolt, pin, weld, or adhesive. Blade support 4A includes longitudinal through bore 4B (disclosed in detail below). Blade support 4A may include retention hole 4C to connect blade holder 4 to housing 2 with, for example pin 22. Further, blade holder 4 has an outer diameter less then that of the inside diameter of clamping member 3 such that blade holder 4 can move freely longitudinally within clamping member 3. At the location of slot 3D in clamping member 3, blade holder 4 is affixed to housing 2 and moves simultaneously with housing 2. Blade holder 4 and housing 2 are attached together and move as one component. The attachment point occurs at the location of slot 3D such that housing 2 and blade holder 4 can move independently of clamping member 3.

As discussed above with respect to FIGS. 1-4B, blade 8 may be electrically conductive and coupled to a power source. Additionally or alternatively, as discussed above with respect to FIGS. 5A and 5B, blade 8 may be made of an electrically-conductive material, and a coil may be provided for inductively heating the blade 8. Further, one or more electrodes may be provided about an inner aperture of the blade holder 4, or otherwise about bore 2E.

Clamping member 3 is generally a hollow cylindrical member with lower end 3A and upper end 3C. Clamping member 3 is freely moveable within the lower part of housing 2. The hollow center of clamping member 3 allows for the positioning and passing therethrough of blade holder 4. Clamping member 3 includes a plurality of retractable arms 3F at lower end 3A. Upper end 3C is defined by two extensions 3B positioned on opposite sides of the cylinder at upper end 3C. Pair of extensions 3B are adapted to form slot 3D. Slot 3D is sized to receive blade holder 4 and gear track 5. Each extension 3B includes retention hole 3E for connecting clamping member 3 to gear track 5 with, for example, pin 23. Slot 3D extends longitudinally along the cylinder and allows for fixation of blade holder 4 to housing 2 (discussed in detail below) without interfering with clamping member 3 ability to traverse within housing 2. Once clamping member 3 is fixedly attached to gear track 5, gear track 5 drives housing 2 up and down over clamping member 3, thereby opening and closing retractable arms 3F.

As discussed above, lower end 3A of clamping member 3 includes a plurality of circumferentially evenly spaced, downwardly facing, outwardly tapered, flexible retractable arms 3F. Retractable arms 3F can be made of elastic material, such as plastic, metal, graphite, or other polymer, that retains its spring-like characteristics. The ends of retractable arms 3F form an opening 3G adapted to engage grooved outer surface IF of open ring ID. When the present invention is in the opened or relaxed position, opening 3G is larger then the outer diameter of open ring ID. Retractable arms 3F extend outwardly in a tapered manner allowing for an open position that allows for positioning of clamping member 3 over the outside diameter of the upper section IJ of shaft IA. As housing 2 is advanced down over retractable arms 3F, the inside diameter of housing 2 engages the tapered edge of retractable arms 3F and results in the closure of retractable arms 3F. When retractable arms 3F are closed, the ends of retractable arms 3F define an opening 3G that is substantially the same diameter as the outer diameter of open ring ID when gap IE of open ring ID is closed. Retractable arms 3F, when closed, cooperate with grooved outer surface IF of open ring ID to crush the foreskin for a hemostasis effect and hold open ring ID in a secure, fixed position prior to the delivery of the axial force of blade 8 to the prepuce foreskin.

As discussed above, retractable arms 3F are fully extended or opened in the unrestrained condition, thereby forming the largest opening diameter 3 G possible. As retractable arms 3F are drawn axially into, for example, housing 2, retractable arms 3F move radially inward, thereby reducing the diameter of opening 3G. Retractable arms 3F are adapted to engaged ring component 1, crush the foreskin, and restrain the movement of ring component 1. Each retractable arm 3F may include inward radial extension 3H, which is preferably curved to fit the contour of grooved outer surface IF, to further enhance the hemostasis effect. Four retractable arms are illustrated, however, any number of arms are acceptable that achieve the desired results discussed in detail below.

One embodiment of the clamping-cutting device 602 can be assembled by aligning retention hole 3E of clamping member 3 with radial hole 5B of gear track 5. A conventional means such as a screw, bolt, or press-fit pin 23 is inserted through retention hole 3E and radial hole 5B of gear track 5, thereby connecting together clamping member 3 and gear track 5. Blade holder 4 is placed within the assembled clamping member 3/gear track 5 and the entire assembly is positioned within through bore 2E of housing 2. Retention hole 4C of blade holder 4 is aligned with assembly hole 2H of housing 2. A conventional means such as a screw, bolt, or press-fit pin 22 is inserted through retention hole 4C of blade holder 4 and assembly hole 2H of housing 2, thereby connecting together housing 2 and blade holder 4. Each lever arm 6 is positioned between pair of projections 21. Semi-circular gears 6A are extended into housing 2A through slot 2D. Gear teeth 6C of semi-circular gears 6A are positioned to engage gears 5 A of gear track 5. A conventional joining device is adapted to pivotally attach lever arm 6 to projection 21, thereby completing the assembly of clamping-cutting device 602.

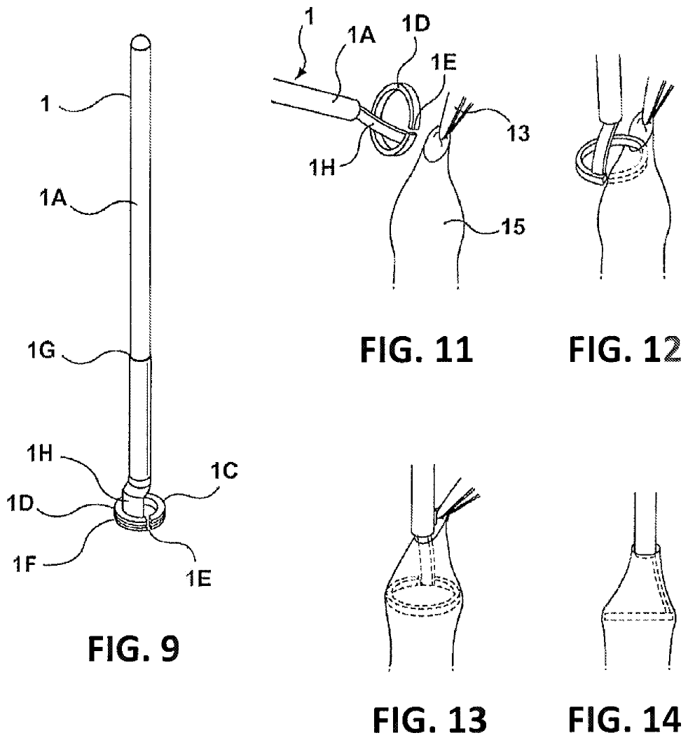

Now turning to FIG. 9, ring component 1 is generally a single piece of malleable, elastic material (such as plastic) with an open ring 1D mounted orthogonally to shaft 1A by curved member 1H, which allows manipulation of open ring 1D within the foreskin. Shaft 1A includes a plurality of diameters to control the insertion of ring component 1 into clamping-cutting device 602. A shaft diameter change can be delineated by a notch or ledge or, as illustrated in FIG. 1, shoulder 1G that acts as a stop of ring component 1 into clamping-cutting device 602. Open ring 1D includes a cutting surface 1C on its top surface being adapted to act as a cutting surface when cutting edge 8 A of blade 8 is pressed down against foreskin interposed between cutting surface 1C and cutting edge 8A. Open ring 1D may also include grooved outer surface IF adapted to engage with inward radial extension 3H of retractable arm 3F to hold ring component in a stationary position relative to housing 2 during the clamping/cutting operation, such that the foreskin is trapped between the grooved outer surface IF and the retractable arms 3F. Further, grooved outer surface 1F will interact with inward radial extension 3H to crush the foreskin against open ring 1D and contemporaneously hold ring component 1 in position while circular blade 8 is delivered to make the incision in the foreskin. Open ring ID includes an opening or gap 1E large enough to allow the thickness of the foreskin to enter. The inner diameter of the open ring ID is large enough to receive a predetermined sized glans and shield the glans or head of the penis from being clamped and/or cut.

One or more electrodes may be provided in shaft 1A, as discussed above with respect to FIGS. 1-5B. Further, an electrode may be provided on top surface 1C, in some embodiments.

FIGS. 11, 12, 13, and 14 demonstrate the insertion of open ring 1D into the neonatal foreskin 15. As discussed above, opening aE allows entry of open ring 1D into foreskin 15. Foreskin 15 is held by an atraumatic forceps 13 while opening 1E is positioned to enter foreskin 15. With a pair of non-traumatic forceps, the foreskin is gently grasped and foreskin 15 is guided into the narrow gap 1E of open ring 1D. With a screw-like motion of shaft 1A of ring component 1, the open edge of open ring ID is advanced in, down, and around the inner aspect of the foreskin. The opening or gap 1E in open ring 1.D allows the open edge to be advanced into the foreskin with a smooth, non traumatic fluid screwing motion. Once open ring 1D is fully inserted and resides just beneath foreskin 15, it can be pushed down slowly toward the glans to free any adhesions. Inside foreskin 15, opening or gap 1E is closed by the elastic nature of the foreskin. The closed open ring 1D, inside the foreskin of the penis, residing just above the glans or tip of the penis, is then used as the compressive surface for any number of clamps, such as inward radial extension 3H, and its top surface acts as a combination glans shield and or cut surface for blade 8 as it cuts the foreskin.

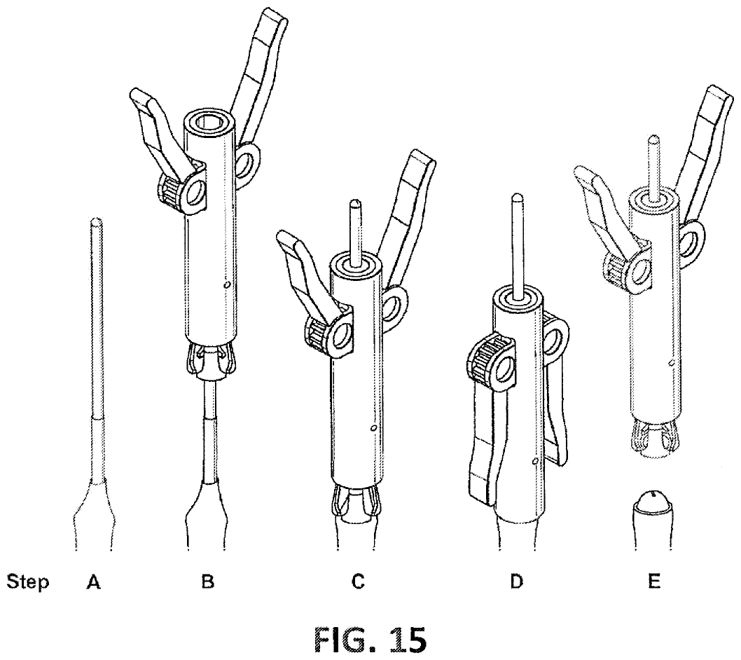

FIG. 15 illustrates a method of using the present invention to perform a neonatal circumcision. Step A: Open ring 1D of ring component 1 is inserted into the foreskin of the penis, as discussed above. Step B: Clamping-cutting device 602 is set in the open position with lever arms 6 rotated up and retractable arms 3F extending fully through the bottom of housing 2. Step C: Clamping-cutting device 602 is inserted onto shaft 1A of ring component 1. Downward movement of clamping-cutting device 602 and housing 2 onto shaft 1A is stopped when shoulder 1G contacts bottom face 5D of gear track. At the termination of such movement, electrodes on shaft 1A align with electrodes about bore 2E. Step D: Lever arms 6 are activated downward to advance housing 2 downward over retractable arms 3F causing the clamping force and delivering the circular blade to the foreskin. More specifically, semi-circular gears 6A of lever arms 6 engage with gear teeth 5A of gear track 5 of clamping-cutting device 602. Housing 2 moves downwardly over retractable arms 3F causing retractable arms 3F to radially close on to and to exert lateral compressive force against the foreskin. Retractable arms 3F exert sufficient lateral compressive force such that inward radial extensions 3H of retractable arms 3F forces the foreskin into grooved outer surface 1F of open ring 1D, thereby clamping the foreskin without any trauma or deformation between the ring component 1 and the clamping-cutting device 102. As housing 2 is advanced even further over the open ring ID, blade 8 is delivered to the top surface of open ring ID and creates the circular incision into the foreskin when open ring ID is within housing 2. Using the top surface IC of open ring ID as the cutting surface that is held in place by the closed retractable arms 3F, blade 8 makes a single, clean, circumferential incision on top of the ring, removing the excess foreskin. Where electrodes and other electrical components are provided, current may be delivered to those electrodes and components before and during the incision. In some embodiments, instead of an incision with a blade, an electrically-conductive cutting component may be placed adjacent to the foreskin, and the electrical current conduction may create the incision. The clamp is left in place for a period of time ensuring adequate crushing and hemostasis. Step E: Lever arms 6 are lifted upward and retractable arms 3F release ring component 1, lifting housing 2 of the clamping- cutting device 102 and lifting blade 8 back up into housing 2 and releasing open ring ID. Shaft 1A with severed foreskin is removed from housing 2. All components of the present invention and byproducts of the operation are thrown away, thereby completing the circumcision.

In a first aspect of the present disclosure, a male circumcision device is disclosed that includes a ring component including a base portion having a first electrode and a shaft having a second electrode, and a cutting component including a body defining an inner aperture, an electrically-conductive cutting implement disposed at a distal end of the cutting component, and a third electrode disposed in the aperture, wherein the cutting implement electrically couples with the first electrode, and the third electrode electrically couples with the second electrode, when the cutting component is in close proximity to the base portion with the shaft inserted in the inner aperture.

In an embodiment of the first aspect, the device further includes a power source electrically coupled to the cutting implement and to the third electrode.

In an embodiment of the first aspect, the first electrode includes an annular, proximal-facing surface. In an embodiment of the first aspect, the second electrode includes an annular electrode disposed about the shaft. In an embodiment of the first aspect, the third electrode includes an annular electrode disposed about the aperture.

In an embodiment of the first aspect, the device further includes a fourth electrode disposed on the shaft and electrically coupled to the cutting component and a fifth electrode disposed in the aperture, wherein the fifth electrode electrically couples with the fourth electrode when the cutting component is in close proximity to the base portion with the shaft inserted in the inner aperture.

In an embodiment of the first aspect, the cutting component is annular about a central axis, wherein the aperture is defined about the axis.

In an embodiment of the first aspect, the shaft defines and extends along a longitudinal ring component axis, wherein the base portion and the first electrode have larger respective diameters than the shaft with respect to the longitudinal ring component axis.

In a second aspect of the present disclosure, a method of circumcising a penis is provided. The method includes inserting a ring of a ring component within the foreskin, the ring component comprising an electrode, placing an axial cutting device about the ring component, the axial cutting device comprising an electrically-conductive cutting component, radially clamping the foreskin against a circumference of the ring, applying an electrical current through the electrode and the electrically-conductive cutting element, and cutting the foreskin while the electrical current is applied.

In an embodiment of the second aspect, the electrode of the ring component is annular and disposed on a proximal surface of the ring. In an embodiment of the second aspect, the electrode of the ring component is annular and disposed on a shaft of the ring component. In an embodiment of the second aspect, the electrically-conductive cutting element is a metallic blade.

In an embodiment of the second aspect, the electrically conductive cutting element comprises a coil and a blade, wherein applying the electrical current through the coil causes the blade to be inductively heated.

In a third aspect of the present disclosure, a male circumcision device is provided. The device includes a ring component including a base portion and a shaft and an electrode disposed on the base portion or the shaft, a cutting component including a body defining an inner aperture, a cutting implement comprising an electrically-conductive portion disposed at a distal end of the cutting component, and a power source for applying an electrical current through the electrode and through the electrically-conductive portion of the cutting component when the cutting component is coupled to the ring component during a cutting operation.

In an embodiment of the third aspect, the electrically-conductive portion of the cutting element comprises a metallic blade. In an embodiment of the third aspect, the cutting element comprises a coil and a blade, wherein applying the electrical current through the coil causes the blade to be inductively heated.

In an embodiment of the third aspect, the electrode is a first electrode and the device further comprises a second electrode, disposed about the aperture of the cutting component and configured to be electrically coupled with the first electrode during the cutting operation.

In an embodiment of the third aspect, the electrode is disposed on the base portion and comprises an annular, proximal-facing surface. In an embodiment of the third aspect, the electrode is disposed on the shaft.

In an embodiment of the third aspect, the shaft defines and extends along a longitudinal ring component axis, wherein the base portion and the electrode have larger respective diameters than the shaft with respect to the longitudinal ring component axis.

While this disclosure has described certain embodiments, it will be understood that the claims are not intended to be limited to these embodiments except as explicitly recited in the claims. On the contrary, the instant disclosure is intended to cover alternatives, modifications and equivalents, which may be included within the spirit and scope of the disclosure. Furthermore, in the detailed description of the present disclosure, numerous specific details are set forth in order to provide a thorough understanding of the disclosed embodiments. However, it will be obvious to one of ordinary skill in the art that systems and methods consistent with this disclosure may be practiced without these specific details. In other instances, well known methods, procedures, components, and circuits have not been described in detail as not to unnecessarily obscure various aspects of the present disclosure.

What is claimed is:

1. A male circumcision device comprising:
a ring component comprising:
a base portion having a first electrode; and
a shaft having a second electrode; and
a cutting component comprising:
a body defining an inner aperture;
an electrically-conductive cutting implement disposed at a distal end of the cutting component; and
a third electrode disposed in the aperture;
wherein the cutting implement electrically couples with the first electrode, and the third electrode electrically couples with the second electrode, when the cutting component is in close proximity to the base portion with the shaft inserted in the inner aperture.

2. The male circumcision device of claim 1, further comprising a power source electrically coupled to the cutting implement and to the third electrode.

3. The male circumcision device of claim 1, wherein the first electrode comprises an annular, proximal-facing surface.

4. The male circumcision device of claim 1, wherein the second electrode comprises an annular electrode disposed about the shaft.

5. The male circumcision device of claim 1, wherein the third electrode comprises an annular electrode disposed about the aperture.

6. The male circumcision device of claim 1, further comprising:
a fourth electrode disposed on the shaft and electrically coupled to the cutting component; and
a fifth electrode disposed in the aperture;
wherein the fifth electrode electrically couples with the fourth electrode when the cutting component is in close proximity to the base portion with the shaft inserted in the inner aperture.

7. The male circumcision device of claim 1, wherein the cutting component is annular about a central axis, wherein the aperture is defined about the axis.

8. The male circumcision device of claim 1, wherein the shaft defines and extends along a longitudinal ring component axis, wherein the base portion and the first electrode have larger respective diameters than the shaft with respect to the longitudinal ring component axis.

9. A method of circumcising a penis, the method comprising:
inserting a ring of a ring component within a foreskin, the ring component comprising an electrode;
placing an axial cutting device about the ring component, the axial cutting device comprising an electrically-conductive cutting component;
radially clamping the foreskin against a circumference of the ring;
applying an electrical current through the electrode and the electrically-conductive cutting component; and
cutting the foreskin while the electrical current is applied.

10. The method of claim 9, wherein the electrode of the ring component is annular and disposed on a proximal surface of the ring.

11. The method of claim 9, wherein the electrode of the ring component is annular and disposed on a shaft of the ring component.

12. The method of claim 9, wherein the electrically-conductive cutting element is a metallic blade.

13. The method of claim 9, wherein the electrically conductive cutting component comprises a coil and a blade, wherein applying the electrical current through the coil causes the blade to be inductively heated.

14. A male circumcision device comprising:
a ring component comprising:
a base portion and a shaft; and
an electrode disposed on the base portion or the shaft;
a cutting component comprising:
a body defining an inner aperture;
a cutting implement comprising an electrically-conductive portion disposed at a distal end of the cutting component; and
a power source for applying an electrical current through the electrode and through the electrically-conductive portion of the cutting component when the cutting component is coupled to the ring component during a cutting operation.

15. The male circumcision device of claim 14, wherein the electrically-conductive portion of the cutting component comprises a metallic blade.

16. The male circumcision device of claim 14, wherein the cutting component comprises a coil and a blade, wherein applying the electrical current through the coil causes the blade to be inductively heated.

17. The male circumcision device of claim 14, wherein the electrode is a first electrode and the device further comprises a second electrode, disposed about the aperture of the cutting component and configured to be electrically coupled with the first electrode during the cutting operation.

18. The male circumcision device of claim 14, wherein the electrode is disposed on the base portion and comprises an annular, proximal-facing surface.

19. The male circumcision device of claim 14, wherein the electrode is disposed on the shaft.

20. The male circumcision device of claim 14, wherein the shaft defines and extends along a longitudinal ring component axis, wherein the base portion and the electrode have larger respective diameters than the shaft with respect to the longitudinal ring component axis.
